(19) United States
(12) Patent Application Publication
(10) Pub. No.: US 2023/0133024 A1
(43) Pub. Date: May 4, 2023

(54) METHOD FOR STORING A BIOGAS IN A TANK AND ASSOCIATED SYSTEM

(71) Applicants: ASSOCIATION POUR LA RECHERCHE ET LE DEVELOPPEMENT DES METHODES ET PROCESSUS INDUSTRIELS - ARMINES, Paris Cedex 06 (FR); ECOLE NATIONALE SUPERIEURE DES MINES DE PARIS, Paris Cedex 06 (FR)

(72) Inventors: Rodrigo Arturo RIVERA TINOCO, PALAISEAU (FR); Maroun NEMER, MASSY (FR)

(21) Appl. No.: 17/917,526

(22) PCT Filed: Mar. 31, 2021
(86) PCT No.: PCT/FR2021/050565

§ 371 Date: Oct. 6, 2022

(30) Foreign Application Priority Data
Apr. 6, 2020 (FR) FR2003423

(51) Int. Cl. C12M 1/107; F25J 1/00

(52) U.S. Cl. CPC C12M 23/36; F25J 1/00; F25J 2210/66; F25J 2215/64; F25J 2290/62

(57) ABSTRACT

The invention essentially concerns a method for storing a biogas in a tank, said method comprising the following steps:

direct contacting of the biogas with a hydrocarbon of the C₃ to C₇ family under conditions allowing at least partial liquefaction of the biogas to obtain a biogas-hydrocarbon mixture that is at least partly liquid, and

storing (S260) the biogas-hydrocarbon mixture in the tank.

### TECHNICAL FIELD

The present invention relates to the field of biogas production and more particularly concerns a method for storing a biogas in a tank.

The invention non-exclusively applies to the production of biogas, to the purification thereof for use in vehicles, to the production of biomethane for a gas network, or compressed or liquefied natural gas, etc.

### PRIOR ART

In known manner, the amount of biogas produced at a given production site is often too small to allow efficient, cost-effective treatment of the biogas on site. Indeed, processes for upgrading biogas are costly and the management of these processes is complex.

Therefore, to reduce treatment costs, biogas produced on a production site is collected to be transported to a centralised treatment site, which affords economies of scale.

For the collection of a biogas, the biogas can be compressed or liquefied to reduce volume and thereby optimise storage and transport thereof. However, the pressure and volume of a biogas are often too high for compression to be taken into consideration. Liquefaction of biogas is also energy-intensive.

In addition, liquefaction of biogas can produce solid particles of carbon dioxide which can subsequently cause clogging in heat exchangers of the biogas treatment system. Indeed, the operating conditions allowing biogas liquefaction also cause solidification of carbon dioxide. For example, at an operating pressure of 20 bar and operating temperature of −50 degrees Celsius, carbon dioxide is in liquid phase but methane is in gas phase. When the operating temperature is lowered to −110 degrees Celsius, the operating pressure still being at 20 bar, methane becomes liquefied and changes from the gas phase to the liquid phase, but the carbon dioxide solidifies and therefore changes from the liquid phase to the solid phase.

The invention sets out to overcome the aforementioned problems and more generally concerns the facilitated storage and optional transport of biogas.

### DESCRIPTION OF THE INVENTION

The present invention concerns a method for storing a biogas in a tank, said method comprising the following steps:
direct contacting of the biogas with a hydrocarbon of the C₃ to C₇ family under conditions allowing at least partial liquefaction of the biogas, to obtain a biogas-hydrocarbon mixture that is at least partly liquid, and
storing the biogas-hydrocarbon mixture in the tank.

Direct contacting of the biogas with the hydrocarbon allows direct heat and mass transfer between the biogas and the hydrocarbon. The biogas is then absorbed and/or solubilised by the liquid hydrocarbon leading to full or partial phase change of the biogas. The biogas-hydrocarbon mixture obtained is therefore biogas absorbed by the liquid hydrocarbon.

The biogas-hydrocarbon mixture obtained has a dew point at a temperature higher than that of the biogas alone, under same pressure conditions.

The hydrocarbon therefore allows a rise in the dew point temperature of the biogas which is therefore able to liquefy at a higher temperature than the solidifying temperature of carbon dioxide. Additionally, preference is given to the hydrocarbon over other carrier agents such as coolants or paraffin since the latter may frost over and are less soluble.

Direct contacting of the biogas with the hydrocarbon therefore allows a biogas to be obtained in liquid form whilst affording reduced energy consumption. In addition, there is a reduced risk of agglomeration and clogging of pipelines by frost particles of biogas carbon dioxide at temperatures of between −56° C. and −125° C. Frost particles may subsist but do not agglomerate in the liquid biogas-hydrocarbon mixture. The biogas is thus easier to transport and to collect.

In one particular embodiment, the storage method further comprises the following steps:
feeding the hydrocarbon of the C₃ to C₇ family into the tank,
injecting the biogas in gaseous form into the tank,
   wherein the direct contacting of the biogas with the hydrocarbon is performed in the tank.

In one particular embodiment, the hydrocarbon is fed into the tank before the biogas injection step.

In one particular embodiment, the biogas is injected into the tank via at least one nozzle, said nozzle being positioned below the hydrocarbon level.

In one particular embodiment, the conditions allowing at least partial liquefaction of the biogas comprise a temperature in the tank of between −110 degrees Celsius and 35 degrees Celsius, and a pressure in the tank of between 1 bar and 1000 bar.

In one particular embodiment, the direct contacting of the biogas with the hydrocarbon is performed outside the tank, in mixing means, the biogas-hydrocarbon mixture obtained then being fed into the tank.

In one particular embodiment, the method comprises a step to control the proportion of biogas in the biogas-hydrocarbon mixture, the injection of the biogas and/or feeding of the hydrocarbon being performed up until the molar composition of the biogas-hydrocarbon mixture in the tank 110 is from 0.00001% to 70% hydrocarbon.

In one particular embodiment, the method further comprises a step to transport the tank for the purpose of taking the biogas out of storage.

In one particular embodiment, the method comprises a step to cool the hydrocarbon before the direct contacting step, the hydrocarbon being in liquid and/or solid form at the direct contacting step, the temperature of the hydrocarbon after the cooling step being a condition allowing at least partial liquefaction of the biogas.

In one particular embodiment, the hydrocarbon is cooled to a temperature of between −110 degrees Celsius and 35 degrees Celsius.

In one particular embodiment, the method comprises a step to compress the biogas, said compression step being performed before the direct contacting step.

The invention further concerns a storage system of a biogas in a tank, the biogas being placed in the tank in direct contact with a hydrocarbon of the C₃ to C₇ family, under conditions allowing at least partial liquefaction of the biogas to obtain a biogas-hydrocarbon mixture that is at least partly liquid, the system comprising the tank, said tank being able to store the biogas-hydrocarbon mixture.

### BRIEF DESCRIPTION OF THE DRAWINGS

Other characteristics and advantages of the present invention will become apparent from the description given below with reference to the appended drawings illustrating an example of embodiment that is in no way limiting. In the Figures:

FIG. 1 schematically illustrates a storage system conforming to an example of embodiment of the invention;

FIG. 2 is a flow chart illustrating the main steps of a storage method conforming to an example of embodiment of the invention; and

FIG. 3 schematically illustrates an ejector of the storage system in FIG. 1.

### DESCRIPTION OF EMBODIMENTS

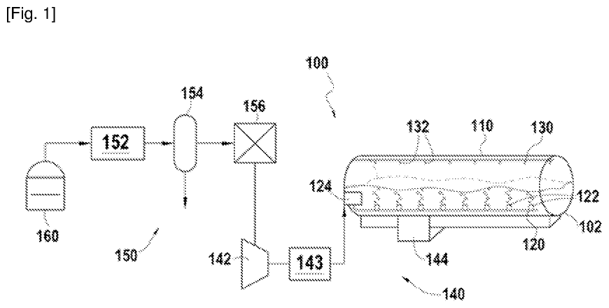

FIG. 1 schematically illustrates a biogas storage system 100 according to an example of embodiment of the invention.

The storage system 100 is able to place the biogas in direct contact with a hydrocarbon to achieve at least partial liquefaction of the biogas and to obtain a biogas-hydrocarbon mixture that is at least partly liquid. The storage system 100 therefore allows storage of the biogas for transport and/or collection thereof.

The term <<biogas>> herein designates an initially gaseous mixture essentially containing methane and carbon dioxide, produced by fermentation of organic matter in the absence of oxygen. The biogas is therefore composed of about 50% methane and 50% carbon dioxide.

Also, the hydrocarbon used is a hydrocarbon of the C₃ to C₇ family. The hydrocarbon is preferably a linear hydrocarbon (an alkane or alkene) but can also be a halogenated hydrocarbon and/or non-liner hydrocarbon (alkane or alkene).

For example, the hydrocarbon used is propane, n-butane, isobutene, n-pentane, isopentane, n-hexane, etc.

The storage system 100 comprises a tank 110, and typically comprises biogas injection means 120 and hydrocarbon feed means 130.

In addition, the storage system 100 may comprise means 140 for managing operating conditions in the tank 110, these management means 140 typically comprising a compressor 142 typically connected to a pre-treatment system 150 of crude biogas, a first heat exchanger 144 (also called <<cooling unit>>) and optionally a second heat exchanger 143, the second heat exchanger 143 typically connecting the compressor 142 to the tank 110.

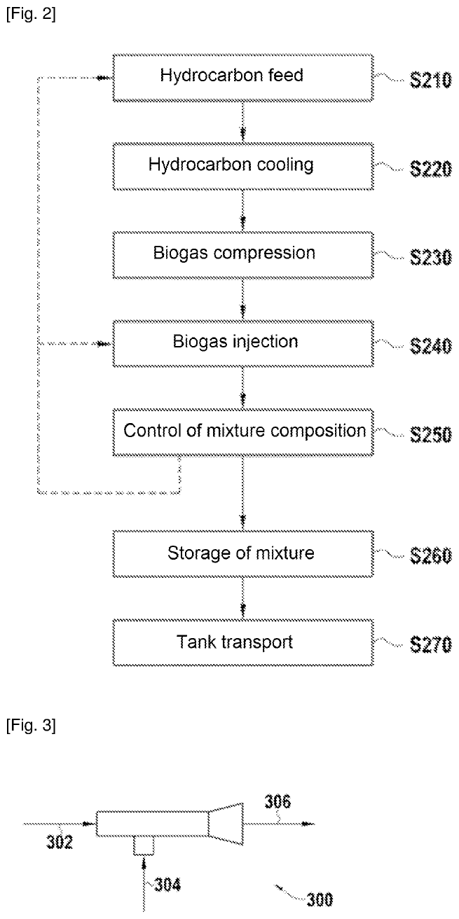

As described in more detail below with reference to FIG. 2, the injection means 120 are able to inject into the tank 110 the biogas that is to be stored. The biogas is typically injected in gaseous form. The injection means 120 are able to be connected to the pre-treatment system 150 of crude biogas, to the second heat exchanger 143 of the management means 140 or to the compressor 142 of the management means 140.

As shown in FIG. 1, the injection means 120 typically comprise one or more nozzles 122, each nozzle being positioned at a lower part of the tank 110 i.e. a part located below the hydrocarbon level after feeding the hydrocarbon into the tank 110. Each nozzle 122 is therefore typically positioned at the bottom 102 of the tank 110.

Each nozzle 122 is also connected to a duct 124 able to connect the injection means 120 to the second heat exchanger 143 of the management means 140, to the compressor 142 of the management means 140 or to the pre-treatment system 150 of crude biogas.

The pre-treatment system 150 is able to be treat crude biogas typically leaving a digester 160 positioned at a production site, before injecting the biogas into the tank 110. The pre-treatment system 150 may comprise one or more items of equipment for example from among the following:
a heat exchanger 152 called <<third heat exchanger>>;
a condenser/separator 154 to reduce the water content of the biogas and extract water from the biogas;
a purifier 156 to reduce the sulfur content of the biogas.

The feed means 130 are able to feed the hydrocarbon of the C₃ to C₇ family into the tank and/or to circulate the hydrocarbon in the tank 110.

The feed means 130 typically comprise one or more nozzles 132. Each nozzle 132 can be positioned at an upper part of the tank 110.

The compressor 142 is able to increase the pressure of the biogas before it is injected into the tank, the pressure of the biogas before entering the compressor typically being close to atmospheric pressure.

The second heat exchanger 143 is able to cool the biogas before it is contacted with the hydrocarbon.

When the hydrocarbon is fed into or stored in the tank 110 before injection of the biogas, the first heat exchanger 144 can cool the hydrocarbon. The first heat exchanger 144 can therefore be positioned inside the tank 110 and can comprise parts of given geometry allowing heat exchange between the hydrocarbon and a coolant. For example, the parts are tubular or rectangular or in the form of plates or may comprise planar surfaces. As a variant, the first heat exchanger 144 can be positioned outside the tank 110 so that it surrounds the tank 110 and can then be of cylindrical shape or oval or rectangular.

The tank 110 is able to store the biogas-hydrocarbon mixture obtained up until the biogas is taken out of storage.

Also, the tank 110 comprising the biogas-hydrocarbon mixture is typically able to be transported via transport means of any type e.g. using a truck or a boat.

As a variant, the biogas injection means 120 and hydrocarbon feed means 130 can be replaced by mixing means positioned outside the tank 110, the mixing means being in the form of an ejector such as ejector 300 shown in FIG. 3. As a variant, the mixing means are in the form of a mixer or duct tube e.g. a concentric mixing tube, Venturi system, mixing tank, etc.

As can be seen in FIG. 3, the ejector 300 comprises a hydrocarbon inlet 302, a biogas inlet 304 and an outlet 306. The biogas inlet 304 is typically connected to the crude biogas pre-treatment system 150, to the second heat exchanger 143 of the management means 140 or to the compressor 142 of the management means 140.

FIG. 2 illustrates a method for storing a biogas in a tank conforming to an example of embodiment of the invention. The storage method is typically implemented by a storage system comprising a tank such as the storage system 100 in FIG. 1 for example.

At step S210, a hydrocarbon of the C₃ to C₇ family is fed into the tank 110 by hydrocarbon feed means 130.

At step S240, the biogas in gaseous form is injected into the tank 110 by the injection means 120. The injected biogas is typically pre-treated by the pre-treatment system 150, this biogas being derived from the digester 160 for example. The temperature of the injected biogas is typically between 10° C. and the storage temperature of the mixture at step S260 described below. As a variant, the biogas is cooled by the pre-treatment system 150 and/or the second heat exchanger 143 so that that the temperature of the injected biogas is typically from −110 to 40 degrees Celsius, or is at ambient temperature.

Injection step S240 of the biogas in gaseous form is typically performed after the hydrocarbon feed step S210. As a variant, the biogas injection step S240 is performed before the hydrocarbon feed step S210, or concomitantly.

The biogas and hydrocarbon are therefore directly placed in contact in the tank 110 under conditions allowing full or partial liquefaction of the biogas, and to obtain a full or partly liquid biogas-hydrocarbon mixture. The hydrocarbon therefore acts as carrier agent for the biogas.

The direct contacting of the biogas with the hydrocarbon allows direct heat and mass transfer between the biogas and the hydrocarbon. The biogas is then absorbed and/or solubilised by the liquid hydrocarbon, which leads to full or partial phase change of the biogas. The biogas-hydrocarbon mixture obtained is therefore biogas absorbed by the liquid hydrocarbon.

The biogas-hydrocarbon mixture obtained has a dew point at a temperature higher than that of the biogas alone under same pressure conditions.

The hydrocarbon therefore allows an increase in the dew point temperature of the biogas which is therefore able to liquefy at a temperature higher than the solidifying temperature of carbon dioxide. Additionally, preference is given to the hydrocarbon over other carrier agents such as coolants or paraffin since the latter may frost over and are less soluble.

The direct contacting of the biogas with the hydrocarbon therefore allows a biogas in liquid form to be obtained, whilst affording reduced energy consumption. In addition, there is a lesser risk of agglomeration and clogging of lines by frost particles of biogas carbon dioxide at temperatures between −56° C. and −125° C. Frost particles may subsist but do not agglomerate in the liquid biogas-hydrocarbon mixture. The biogas is therefore easier to transport and to collect.

One of the conditions allowing at least partial liquefaction of the biogas is the temperature of the hydrocarbon at the time of direct contact thereof with the biogas. The temperature of the hydrocarbon when it is fed into the tank is therefore typically lower than 35° C.

The hydrocarbon can therefore be cooled by the first heat exchanger 144 to a temperature of between −110 degrees Celsius and 35 degrees Celsius, for example to −80 degrees Celsius.

In addition, for reasons of solubility, the hydrocarbon is in liquid and/or solid form at the time of direct contacting between the biogas and the hydrocarbon. The liquid phase is preferred since the hydrocarbon is then easier to mix.

Another condition allowing at least partial liquefaction of the biogas is the operating pressure inside the tank 110_{[JMY1]}. The operating pressure inside the tank 110 is typically equal to the pressure of the injected biogas to avoid having to add other components.

Therefore, the method may comprise a compression step S230 of the biogas by the compressor 142, performed before the injection step of the biogas into the tank 110. The pressure of the compressed biogas can then by between 1 bar and 1000 bar, and for example is 20 bar.

The operating pressure in the tank 110 is between 1 bar and 1000 bar, and for example is 20 bar.

The proportion of biogas in the biogas-hydrocarbon mixture can be controlled at step S250, the injection of the biogas and/or feeding of hydrocarbon then being performed up until the molar composition of the hydrocarbon in the biogas-hydrocarbon mixture in the tank 110 lies in the range from a few traces of hydrocarbon (i.e. 0.00001% hydrocarbon) up until a maximum of about 70% hydrocarbon, the remainder of the biogas-hydrocarbon mixture being biogas. For example, the molar composition of the biogas-hydrocarbon mixture in the tank 110 is 70% hydrocarbon and 30% biogas.

The proportion of biogas in the biogas-hydrocarbon mixture is typically controlled by one or more sensors such as a weighing device and/or flowmeter.

When the operating temperature is −80 degrees Celsius and the operating pressure 20 bar, the biogas-hydrocarbon mixture is fully liquid.

When the hydrocarbon is in the tank 110 before step S230 of injecting the biogas, the biogas is typically injected into the tank 110 via the nozzle(s) 122 positioned below the hydrocarbon level. This type of injection of the biogas allows optimal mixing of the biogas with the hydrocarbon.

As a variant, when the biogas is injected into the tank 110 before the hydrogen feed step S210, the hydrocarbon can be sprayed in the form of liquid droplets by means of nozzles 132.

The direct contacting of the biogas and hydrocarbon can be improved through the presence of bulk or structured lining means in the tank 110 allowing the creation of a liquid film in contact with the biogas.

As a variant or in addition, the direct contacting of the biogas and hydrocarbon can be improved by pumping the (non-saturated) biogas-hydrocarbon mixture already formed in the tank 110, followed by injection or spraying of the pumped mixture into the tank 110. Since heat and mass transfer is limited by contact between the hydrocarbon and biogas, this operation allows liquefaction of a greater amount of biogas.

As a variant or in addition, the direct contacting of the biogas and hydrocarbon can be improved through the presence of any means allowing contact between a liquid and a gas, such as one or more bubbling zones inside the tank 110.

The formation of the biogas-hydrocarbon mixture inside the tank 110 can be diabatic or adiabatic transformation. The amount of biogas to be stored, the chosen temperature and pressure for the mixing operation in the tank 110 will allow the defining of the diabatic or adiabatic process to allow optimisation of biogas absorption and storage in the hydrocarbon.

As a variant, step S210 to feed the hydrocarbon into the tank 110 and step S240 to inject the biogas into the tank are not implemented and are replaced by a direct contacting step of the biogas and hydrocarbon outside the tank 110, in the mixing means of the storage system 100, to obtain a biogas-hydrocarbon mixture, this direct contacting step being followed by a step to feed the biogas-hydrocarbon mixture obtained into the tank 110.

At this direct contacting step of the biogas with the hydrocarbon outside the tank 110, contacting is also performed under conditions allowing at least partial liquefaction of the biogas to obtain a biogas-hydrocarbon mixture that is at least partly liquid. Contacting outside the tank 110 is therefore typically performed under the same conditions of temperature and pressure as for contacting within the tank 110 and produces the same effects.

Direct contacting outside the tank 110 is typically performed in an ejector such as ejector 300 schematically illustrated FIG. 3. The direct contacting step then comprises feeding the hydrocarbon into the ejector 300 via the hydrocarbon inlet 302 and injecting the biogas into the ejector 300 via the biogas inlet. 304, feeding of the hydrocarbon possibly being performed before, after or during injection of the biogas.

The hydrocarbon feed is typically pumped from the tank 110. In addition, the injected biogas is typically pre-treated by the pre-treatment system 150, cooled by the second heat exchanger 143 and/or compressed by the compressor 142.

Direct contacting between the hydrocarbon and biogas is promoted by the high-speed flow of the biogas, of the hydrocarbon and/or of the biogas-hydrocarbon mixture into the body of the ejector 300. The biogas-hydrocarbon mixture then leaves the ejector 300 via outlet 306 and is fed into the tank 110.

As a variant, contacting is performed using a mixer or duct tube e.g. a concentric mixing tube, Venturi system, mixing tank, etc.

In this variant, the proportion of biogas in the biogas-hydrocarbon mixture can also be controlled so that the molar composition of the hydrocarbon in the biogas-hydrocarbon mixture in the tank 110 lies in a range from a few traces of hydrocarbon up to a maximum of about 70% hydrocarbon, the remainder of the biogas-hydrocarbon mixture being biogas. For example, the molar composition of the biogas-hydrocarbon mixture in the tank 110 is 70% hydrocarbon and 30% biogas.

The biogas-hydrocarbon mixture is stored at step S260 in the tank 110.

The tank 110 may additionally be transported via the transport means at step S370, for the biogas to be taken out of storage (taking out of storage being the operation of separating the biogas from the hydrocarbon). Therefore, the transport means can transport the tank 110 from the production site to a centralised biogas treatment site where the biogas is collected (i.e. removed from the tank and optionally purified to obtain biomethane).

1. A method for storing a biogas in a tank, said method comprising the following steps:
direct contacting of the biogas with a hydrocarbon of the C₃ to C₇ family under conditions allowing at least partial liquefaction of the biogas, to obtain a biogas-hydrocarbon mixture that is at least partly liquid, and
storing the biogas-hydrocarbon mixture in the tank.

2. The storage method according to claim 1, further comprising the following steps:
feeding the hydrocarbon of the C₃ to C₇ family into the tank,
injecting the biogas in gaseous form into the tank,
wherein the direct contacting of the biogas with the hydrocarbon is performed in the tank.

3. The storage method according to claim 2, wherein the hydrocarbon is fed into the tank before the biogas injection step.

4. The storage method according to claim 3, wherein the biogas is injected into the tank via at least one nozzle, said nozzle being positioned below the hydrocarbon level.

5. The storage method according to claim 2, wherein the conditions allowing at least partial liquefaction of the biogas comprise a temperature in the tank of between −110 degrees Celsius and 35 degrees Celsius, and a pressure in the tank of between 1 bar and 1000 bar.

6. The storage method according to claim 1, wherein the direct contacting of the biogas with the hydrocarbon is performed outside the tank, in mixing means, the biogas-hydrocarbon mixture obtained then being fed into the tank.

7. The storage method according to claim 1 comprising a step to control the proportion of biogas in the biogas-hydrocarbon mixture, the injection of the biogas and/or feeding of the hydrocarbon being performed up until the molar composition of the biogas-hydrocarbon mixture in the tank is from 0.00001% to 70% hydrocarbon.

8. The storage method according to claim 1 further comprising a step to transport the tank for the purpose of taking the biogas out of storage.

9. The storage method according to claim 1 comprising a step to cool the hydrocarbon before the direct contacting step, the hydrocarbon being in liquid and/or solid form at the direct contacting step, the temperature of the hydrocarbon after the cooling step being a condition allowing at least partial liquefaction of the biogas.

10. The storage method according to claim 9, wherein the hydrocarbon is cooled to a temperature of between −110 degrees Celsius and 35 degrees Celsius.

11. The storage method according to claim 1 comprising a step to compress the biogas, said compression step being performed before the direct contacting step.

12. A storage system of a biogas in a tank, the biogas being placed in direct contact in the tank with a hydrocarbon of the C₃ to C₇ family under conditions allowing at least partial liquefaction of the biogas to obtain a biogas-hydrocarbon mixture that is at least partly liquid, the system comprising the tank, said tank being able to store the biogas-hydrocarbon mixture.
